(19) United States
(12) Patent Application Publication
(10) Pub. No.: US 2026/0096741 A1
(43) Pub. Date: Apr. 9, 2026

(54) SYSTEM AND METHOD FOR RECEIVER COIL PLACEMENT GUIDANCE FOR INTRAOPERATIVE MRI

(71) Applicant: SYNAPTIVE MEDICAL INC., Mississauga (CA)

(72) Inventors: Chad Tyler HARRIS, Toronto (CA); Alex Gyles PANTHER, Toronto (CA); Cameron Anthony PIRON, Toronto (CA); Gal SELA, Toronto (CA)

(21) Appl. No.: 19/345,290

(22) Filed: Sep. 30, 2025

Related U.S. Application Data
(60) Provisional application No. 63/704,590, filed on Oct. 8, 2024.

(51) Int. Cl. A61B 5/055; A61B 5/00; G01R 33/34

(52) U.S. Cl. CPC A61B 5/055; A61B 5/742; G01R 33/34084

(57) ABSTRACT

A system and method for receiver coil placement for an intraoperative magnetic resonance imaging (MRI) system. A receiver coil array or loop has an identification marker or markers that can be located with a navigation system. The relative coil geometries are simulated and an SNR map is generated. The simulated SNR map is super-imposed onto the image used for navigation and displayed to the user. This map can be updated in real-time as the placement of the receiver coils are adjusted to allow optimization of SNR over a particular region of interest.

### CROSS REFERENCE TO RELATED APPLICATIONS

This application claims priority to and the benefit of U.S. Provisional Application Ser. No. 63/704,590, entitled “SYSTEM AND METHOD FOR RECEIVER COIL PLACEMENT GUIDANCE FOR INTRAOPERATIVE MRI”, filed on Oct. 8, 2024, the disclosure of which is incorporated herein by reference in its entirety.

### FIELD OF INVENTION

The field of the invention is systems and methods for magnetic resonance imaging (“MRI”), in particular radiofrequency (RF) coils for MRI.

### BACKGROUND

The placement of receiver coils for intraoperative magnetic resonance imaging (MRI) is cumbersome. Depending on the procedure, the use of static coils is impossible, requiring flexible surface arrays, known as “flex coils” to be used.

Additionally, a new type of flexible coil has recently been developed known as a high-impedance coil (HIC). HICs do not easily couple with one-another, allowing the user to place them freely, without concern of reduced signal-to-noise ratio (SNR) from mutual coupling. However, regardless of receiver coil type, one would like to maximize the achievable SNR from the set of coils over the particular region of interest (ROI). This is not easily done, since the SNR of each loop (or the combined SNR of the array) is related to the geometry of the receiver coil loops with respect to the patient and with respect to the direction of the main magnetic field.

Therefore, as the relative positions of the loops change, or the positions of the loops with respect to the anatomy of interest change, or the orientation of the loops with respect to the main magnetic field direction change, the maximum achievable SNR will change. This relationship becomes more complicated the more flexibility in coil placement the user has.

There is a need for optimized receiver coil placement for MRI.

### BRIEF DESCRIPTION OF THE DRAWINGS

FIG. 1 is a diagram illustrating exemplary surgery systems and equipment.

FIG. 2 is a diagram illustrating an exemplary system of the present disclosure.

FIG. 3 is a diagram illustrating an exemplary workflow of the present disclosure.

### SUMMARY

A system and method for optimal placement of a receiver coil for an intraoperative magnetic resonance imaging (MRI) system is provided.

In some aspects, a coil placement system for an intraoperative MRI is provided, the system including a receiver coil, an identification marker attached to the receiver coil, a detection system for locating the identification marker, a computing device communicating with the detection system, for calculating a coil position from the location of the identification marker; a coil geometry relative to a patient and an MRI magnetic field and a signal-to-noise ratio (SNR) map from the coil geometry, and a display for presenting an image of the SNR map superimposed onto a surgical navigation image. The computing device updates the SNR map on the display in real-time as the receiver coil is placed.

In some aspects, a method for placing a receiver coil of an intraoperative MRI is provided, the method including placing the receiver coil with an attached identification marker in a vicinity of a patient, detecting the location of the identification marker with a detection system, calculating a coil position and a coil geometry relative to the patient and an MRI magnetic field with a computing device, calculating a simulated SNR map from the coil geometry with the computing device and superimposing the SNR map onto a surgical navigation image on a display.

### DETAILED DESCRIPTION

Current approaches to intraoperative MRI coil placement have typically relied on preoperative calibration of receiver coil arrays. In these methods, a coil array is positioned relative to a patient in a known and reproducible orientation, and signal-to-noise ratio (SNR) characteristics are measured in advance using phantom studies or precomputed field simulations. The resulting SNR maps are then stored and later retrieved during surgery, but no real-time adjustment is made as the coil is repositioned intraoperatively. Such static SNR data may not accurately reflect changes in coil proximity to anatomy or variations in the MRI magnetic field during the procedure.

Other techniques employ optical or electromagnetic tracking systems to locate the physical position of a receiver coil in the operating room. In optical tracking approaches, passive or active markers affixed to the coil are detected by ceiling-mounted cameras, and the coil's coordinates are registered to a patient reference frame. Electromagnetic tracking similarly uses sensors embedded in the coil housing to determine position and orientation relative to a field generator. While these methods can report coil geometry in real time, they typically focus on spatial localization alone and do not compute or visualize corresponding SNR distributions.

A further class of systems integrates coil tracking data with surgical navigation platforms, enabling surgeons to see the coil's outline superimposed on a pre-acquired anatomical image. These systems may allow manual selection of predefined SNR contours or color maps, but they do not recalculate SNR continuously as the coil moves. Instead, SNR estimates remain based on initial positioning, without accounting for dynamic shifts in coil-to-tissue distance or changes in the MRI magnetic field gradients.

Some research prototypes have explored combining coil tracking with electromagnetic field simulations to update expected sensitivity profiles during surgery. Such approaches often require high computational resources or simplified coil models, resulting in delayed updates or low-resolution maps. Moreover, integration of these simulated SNR profiles into a real-time surgical display has been limited, and user interaction generally involves separate windows for navigation and coil performance metrics.

However, none of these approaches have provided a comprehensive solution that combines the features described in this disclosure.

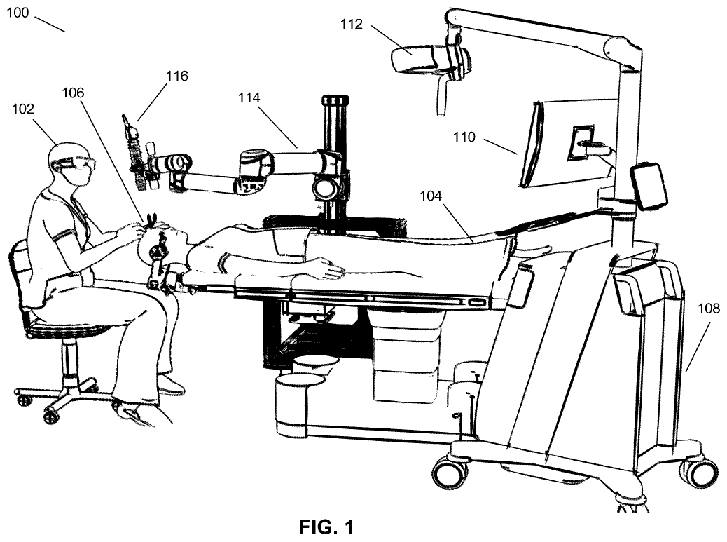

Referring to FIG. 1, an exemplary navigation system 100 which may be used in surgery is shown. A surgeon 102 conducts a surgery on a patient 104 in an operating room environment. The medical navigation system 100 is illustrated including an equipment tower 108, supporting a computing device (not shown) such as a desktop computer, as well as one or more displays 110 connected to the computing device for displaying images provided by the computing device.

Equipment tower 108 also supports a tracking system 112. Tracking system 112 is generally configured to track the positions of one or more tracking markers 106 mounted on an access port (not shown), any other surgical tools, or any combination thereof. Such markers, also referred to as fiducial markers, may also be mounted on patient 104, for example at various points on the head of patient 104. Tracking system 112 may therefore include a camera (e.g. a stereo camera) and a computing device (either the same device as mentioned above or a separate device) configured to locate the fiducial markers in the images captured by the camera, and determine the spatial positions of those markers within the operating theatre. The spatial positions may be provided by tracking system 112 to the computing device in equipment tower 108 for subsequent use.

The nature of the markers and the camera are not particularly limited. For example, the camera may be sensitive to infrared (IR) light, and tracking system 112 may include one or more IR emitters (e.g. IR light emitting diodes (LEDs)) to shine IR light on the markers. In other examples, marker recognition in tracking system 112 may be based on radio frequency (RF) radiation, visible light emitted from devices such as pulsed or un-pulsed LEDs, electromagnetic radiation other than IR or visible light, and the like. For RF and EM-based tracking, each object can be fitted with markers having signatures unique to that object, and tracking system 112 can include antennae rather than the above-mentioned camera. Combinations of the above may also be employed.

Each tracked object generally includes three or more markers fixed at predefined locations on the object. The predefined locations, as well as the geometry of each tracked object, are configured within tracking system 112, and thus tracking system 112 is configured to image the operating theatre, compare the positions of any visible markers to the pre-configured geometry and marker locations, and based on the comparison, determine which tracked objects are present in the field of view of the camera, as well as what positions those objects are currently in. An example of tracking system 112 is the “Polaris” system available from Northern Digital Inc.

Also shown in FIG. 1 is an automated articulated arm 114, also referred to as a robotic arm, carrying an external scope 116 (i.e. external to patient 104). External scope 116 may be positioned over access port by robotic arm 114, and may capture images of the brain of patient 104 for presentation on display 110. The movement of robotic arm 114 to place external scope 116 correctly over access port may be guided by tracking system 112 and the computing device in equipment tower 108. The images from external scope 116 presented on display 110 may be overlaid with other images, including images obtained prior to the surgical procedure. The images presented on display 110 may also display virtual models of surgical instruments present in the field of view of tracking system 112 (the positions and orientations of the models having been determined by tracking system 112 from the positions of the markers mentioned above).

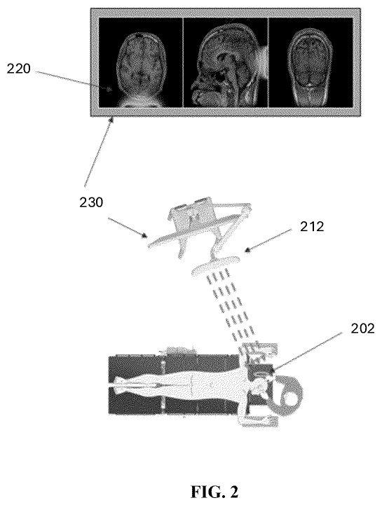

FIG. 2 is a diagram illustrating an exemplary system. According to the present disclosure, a receiver coil array or loop 202 of an MRI has an attached identification marker or markers 106 that can be located with a detection system 212 such as the tracking system 112 presented above. As the receiver coil array or loop 202 is placed, the coil geometry with respect to the patient and with respect to the direction of the main magnetic field is simulated by a computing device in communication with the detection system, using the identification marker or markers 106. Since the SNR of each loop of the receiver coil 202 (or the combined SNR of the array) is related to the geometry of the receiver coil loops with respect to the patient and with respect to the direction of the main magnet field, a signal-to-noise (SNR) map is generated by the computing device from the simulation of the coil geometry. The SNR map is super-imposed onto the image used for navigation 220 and displayed to the user on a display screen 230 as shown in FIG. 2.

According to FIG. 2, a surgeon places the receiver coil 202. The detection system 212 identifies the location of the receiver coil 202 via one or more identification markers attached to the receiver coil and simulated SNR maps are calculated and superimposed on the stereotactic images for the surgeon to assess 220. This map can be updated in real-time as the placement of the receiver coil 202 is adjusted to allow optimization of SNR over a particular region of interest.

In another embodiment, the simulated SNR maps and stereotactic images 220 are displayed to the user via augmented reality (AR). In this case the SNR map is overlayed on a real-time video image of the actual patient rather than the scans used for surgical navigation. If the video system is a two-camera stereoscopic display then the AR overlay can be rendered from different perspective for each camera, presenting the overlay in three dimensions, with depth, when viewed on a three-dimensional display.

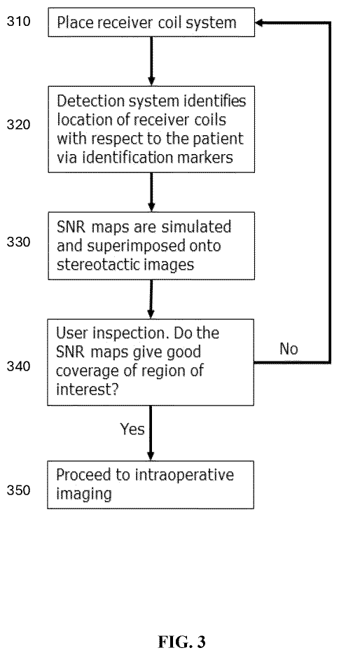

FIG. 3 is a diagram illustrating an exemplary workflow flow chart. According to FIG. 3, the workflow initiates with placing a receiver coil system 310, including a receiver coil array or loop, in the vicinity of a patient. A detection system identifies the location of the receiver coils with respect to the patient and an MRI magnetic field via identification markers 320 attached to the receiver coil array or loop.

According to FIG. 3, the SNR maps are simulated by a computing device and superimposed onto stereotactic images 330 on a display. The next step is user inspection 340 wherein the SNR maps are assessed to determine whether they provide good coverage over the region of interest. If the response is No, the system loops back to the start 310. If the response is Yes, the workflow will enable the process to proceed with intraoperative imaging 350.

In another embodiment, the inspection and assessment of the SNR maps is done automatically, and feedback is provided to the user. This feedback could be provided over multiple sensory modalities, for example, a combination of visual, auditory, and haptic feedback.

According to the disclosure, based on the particular region of interest, the image used for navigation, and the receiver array/loops selected for use, the system can provide suggested orientations of the receivers to maximize SNR.

The identification marker could be at one or two locations on a rigid array, or it could cover the entire extent of a flexible array/loop or be located at specific points of interest so long as an accurate representation of the coil location can be achieved.

The identification marker could be implemented using optical landmarks, RFID (radiofrequency identification) tags, or any other marker used for stereotaxy.

A coil placement system for an intraoperative MRI is disclosed. The system comprises a receiver coil, an identification marker attached to the receiver coil, a detection system for locating the identification marker, a computing device communicating with the detection system. The computing device is configure for calculating a coil position from a location of the identification marker, a coil geometry relative to a patient and an MRI magnetic field and an SNR map from the coil geometry.

According to the disclosure, the system further comprises a display for presenting an image of the SNR map superimposed onto a surgical navigation image. Furthermore, the computing device of the system updates the SNR map on the display in real-time as the receiver coil is placed.

According to the disclosure, the receiver coil comprises a receiver coil array or a coil loop. The computing device of the system further comprises an assessment and feedback component, for assessing the SNR map and providing a feedback to a user.

According to the disclosure, the feedback component comprises at least one of a visual, an auditory and a haptic feedback. The identification marker of the system comprises a plurality of markers on a rigid coil array or a flexible coil array.

According to the disclosure, the identification marker comprises a marker covering an entire extent of a flexible coil array. The identification marker of the system comprises an optical marker or a radiofrequency identification tag.

According to the disclosure, a method for placing a receiver coil of an intraoperative MRI is disclosed. The method comprising the steps of placing the receiver coil with an attached identification marker in a vicinity of a patient, detecting a location of the identification marker with a detection system, calculating a coil position and a coil geometry relative to the patient and an MRI magnetic field with a computing device, calculating a simulated SNR map from the coil geometry with the computing device, and superimposing the SNR map onto a surgical navigation image on a display.

According to the disclosure, placing the receiver coil of the method comprises placing a receiver coil array or a coil loop. The method further comprises assessing the SNR map with the computing device and providing a feedback to a user for placing the receiver coil.

According to the disclosure, the feedback of the method comprises at least one of a visual, an auditory and a haptic feedback to a user. The identification marker of the method comprises a plurality of markers on a rigid coil array or a flexible coil array.

According to the disclosure, the identification marker of the method comprises a marker covering an entire extent of a flexible coil array. The identification marker of the method comprises an optical marker or a radiofrequency identification tag.

While some embodiments or aspects of the present disclosure may be implemented in fully functioning computers and computer systems, other embodiments or aspects may be capable of being distributed as a computing product in a variety of forms and may be capable of being applied regardless of the machine or computer readable media used to affect the distribution.

At least some aspects disclosed may be embodied, at least in part, in software. That is, some disclosed techniques and methods may be carried out in a computer system or other data processing system in response to its processor, such as a microprocessor, executing sequences of instructions contained in a memory, such as read-only memory (ROM), volatile random access memory (RAM), non-volatile memory, cache or a remote storage device.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof configured to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference. The disclosed or illustrated tasks can be distributed across multiple processors or computing devices of a computer system, including computing devices that are geographically distributed.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

The specific embodiments described above have been shown by way of example and understood is that these embodiments may be susceptible to various modifications and alternative forms. Further understood is that the claims are not intended to be limited to the forms disclosed, but rather to cover all modifications, equivalents, and alternatives falling within the spirit and scope of this disclosure. While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

1. A coil placement system for an intraoperative MRI, the system comprising:
a receiver coil;
an identification marker attached to the receiver coil;
a detection system for locating the identification marker;
a computing device communicating with the detection system, for calculating:
a coil position from a location of the identification marker;
a coil geometry relative to a patient and an MRI magnetic field; and
an SNR map from the coil geometry; and
a display for presenting an image of the SNR map superimposed onto a surgical navigation image;
wherein the computing device updates the SNR map on the display in real-time as the receiver coil is placed.

2. The system of claim 1, wherein the receiver coil comprises a receiver coil array or a coil loop.

3. The system of claim 1, wherein the computing device comprises an assessment and feedback component, for assessing the SNR map and providing a feedback to a user.

4. The system of claim 3, wherein the feedback component comprises at least one of a visual, an auditory and a haptic feedback.

5. The system of claim 1, wherein the identification marker comprises a plurality of markers on a rigid coil array or a flexible coil array.

6. The system of claim 1, wherein the identification marker comprises a marker covering an entire extent of a flexible coil array.

7. The system of claim 1, wherein the identification marker comprises an optical marker or a radiofrequency identification tag.

8. A method for placing a receiver coil of an intraoperative MRI, the method comprising:
placing the receiver coil with an attached identification marker in a vicinity of a patient;
detecting a location of the identification marker with a detection system;
calculating a coil position and a coil geometry relative to the patient and an MRI magnetic field with a computing device;
calculating a simulated SNR map from the coil geometry with the computing device; and
superimposing the SNR map onto a surgical navigation image on a display.

9. The method of claim 8, wherein placing the receiver coil comprises placing a receiver coil array or a coil loop.

10. The method of claim 8, further comprising assessing the SNR map with the computing device and providing a feedback to a user for placing the receiver coil.

11. The method of claim 10, wherein the feedback comprises at least one of a visual, an auditory and a haptic feedback to a user.

12. The method of claim 8, wherein the identification marker comprises a plurality of markers on a rigid coil array or a flexible coil array.

13. The method of claim 8, wherein the identification marker comprises a marker covering an entire extent of a flexible coil array.

14. The method of claim 8, wherein the identification marker comprises an optical marker or a radiofrequency identification tag.
